(19) United States
(12) Patent Application Publication
(10) Pub. No.: US 2021/0049271 A1
(43) Pub. Date: Feb. 18, 2021

(54) SYSTEMS AND METHODS FOR DETECTING UNAUTHORIZED FILE ACCESS

(71) Applicants: Arizona Board of Regents of Behalf ot the University of Arizona, Tucson, AZ (US); Brigham Young University, Provo, UT (US)

(72) Inventors: Joseph Valacich, Tucson, AZ (US); Jeffrey Jenkins, Provo, UT (US)

(21) Appl. No.: 17/051,166

(22) PCT Filed: May 2, 2019
(86) PCT No.: PCT/US2019/030369

§ 371 Date: Oct. 27, 2020

Related U.S. Application Data
(60) Provisional application No. 62/665,744, filed on May 2, 2018.

(51) Int. Cl. G06F 21/55; G06F 21/62

(52) U.S. Cl. CPC G06F 21/554; G06F 2221/034; G06F 21/6218

(57) ABSTRACT

Systems and methods for detecting unauthorized data access on a file system are disclosed. These systems and methods do so by compiling a database of user behaviors associated with unauthorized data access, detecting a user's behavior on a networked computing device, scoring the user's behavior against the database of user behaviors, and notifying an administrator, based on the scoring, that the user's behaviors are indicative of unauthorized data access. The systems and methods also create an EFSS or other FMS solution simulation to train its algorithm on which behaviors are likely to be unauthorized and monitors such behaviors as mouse cursor speed and trajectory.

### RELATED APPLICATIONS

This application claims the benefit of U.S. Provisional Application No. 62/665,744, filed May 2, 2018.

### FIELD OF THE INVENTION

The present invention relates to systems and methods for computer-implemented identification of unauthorized data access to a computer or file system. More specifically, the systems and methods analyze behavior and movement anomalies to determine unauthorized access.

### BACKGROUND OF THE INVENTION

Organizations utilize a variety of file management systems (FMS) to enable trusted employees and contractors access to the large troves of data stored in multiple locations (e.g., cloud, on premise servers, and so on). An FMS is a type of software that manages data files and access controls to those files in a computer system. There are different types of FMS depending on where the data resides. A cloud-based FMS, also known as Enterprise File Synchronization and Sharing (EFSS) is a type of service that allows the management of data files remotely over the Internet and permits access to the files to those who have been granted access, authorized users. An EFSS allows an organization to create, edit, delete, and share various files (e.g., text documents, spreadsheets, presentations, graphics, images, videos, code repositories, etc.) in an organized manner with individuals within the organization. EFSS is now very common in most organizations. On the supply side, the EFSS marketplace is heavily saturated with over 100 vendors. The largest EFSS by market share are Amazon, Dropbox, Google Drive, Box, and Microsoft's OneDrive. Gartner predicts that by the end of 2018, 90% of enterprise content, collaboration, file storage and backups will take place on EFSS.

Now, with data proliferating on EFSS services and other FMS services that are outside the organizations typical network, the problem of access control has become practically impossible to manage. In order to better manage authorized access, and in turn unauthorized access, we propose developing a novel capability, the near real-time detection of possible unauthorized data access (UDA) events, which can be easily integrated into existing EFSS and other FMS solutions. This application outlines the development of a novel method for improving the classification of both malicious and non-malicious insider threats within an operational EFSS or other types of FMS environments.

All modern organizations are threatened by the menace of UDA. UDA refers to the unsanctioned access of an organization's data and information resources (e.g., customer records, intellectual property, trade secrets, etc.) by employees, contractors or outsiders. 69% of organizations reported one or more unauthorized theft or corruption of data by insiders in 2016. Reports suggest that there are two basic types of individuals that engage in UDA. One, non-malicious individuals engage in UDA out of curiosity, boredom or even the desire for recognition. Two, malicious individuals engaged in criminal activities that are motivated by monetary gains or revenge against the organization. Non-malicious individuals tend to work on their own while the malicious actors have been known to hire hackers on the dark web that help them identify and sell the valuable data.

Security experts believe that most UDA events are unknown to the organization, and many of those that are known, go unreported out of fear of how such disclosure will damage the organization's reputation. Nevertheless, highly visible reports of UDA are numerous and seemingly unending. This holds true for governmental agencies and contractors. Edward Snowden, for example, engaged in one of the highest profile acts of UDA. In 2013, Snowden accessed and stole upwards of 1.7 million files while as a contractor for the NSA. More recently, the intelligence contractor Reality Winner printed and sent classified reports on Russia's interference in the 2016 election to the news outlet, The Intercept. Winner was caught, not because of access control logs or file tracking technology, but due to unnoticeable marks left on the document by the printer that were published online by the Intercept. These marks identified the serial number of the printer Winner used.

Businesses are also not immune to UDA insider threats. In fact, security experts are adamant that malicious insiders pose the greatest threat for data security threats. The problem of UDA is so pervasive that Carnegie Mellon University has set up a database which tracks over 1,000 publicly available insider incidents in the US. Clearly, UDA is a massive problem that can have a wide range of deleterious effects on virtually any organization.

Keeping tight access control on sensitive data is now harder than ever. Massive data centers share millions of data files with both employees and contractors that are hosted on large cloud architectures. With the onset of Agile and DevOps practices, teams change more rapidly than ever before. Not only do the administrators have to manage access to the ever-growing quantity of data, but they must also maintain proper access to the rapidly changing organization and teams. One study reports that 62% of business-users have access to data they probably should not be able to view. Maintaining proper access control to sensitive data is an unsolved problem.

To accomplish this goal, described herein are systems and methods to detect and report unauthorized file access via behavioral anomalies (e.g., non-typical patterns of accessing files) and movement anomalies reflected in the way people move their mouse or other HCI device when navigating and operating within the EFSS. From the systems and methods, intentions, actions, and behavior may be inferred via changes in the way people move their HCI device. In this manner, the system and method identifies behavioral and movement patterns by tracking both authorized and unauthorized events to train a machine learning algorithm.

### SUMMARY OF THE INVENTION

It is therefore an object of the invention to disclose systems and methods for predictive identification of unauthorized file access that do so by compiling a database of user behaviors associated with unauthorized data access, detecting a user's behavior on a networked computing device, scoring the user's behavior against the database of user behaviors, and notifying an administrator, based on the scoring, that the user's behaviors are indicative of unauthorized data access.

It is another object of the invention to disclose systems and methods for predictive identification of unauthorized file access that do so by creating an EFSS simulation to compile the database of user behaviors.

It is yet another object of the invention to disclose systems and methods for predictive identification of unauthorized file access that do so by monitoring mouse cursor x- and y-positions and associated timestamps.

### BRIEF DESCRIPTION OF THE FIGURES

FIG. 1 shows a diagram of the hardware utilized, in accordance with an embodiment of the invention;

FIG. 2 shows a flow chart of the software pathway, in accordance with an embodiment of the invention;

FIG. 3 shows a graphic user interface of a simulated EFSS, in accordance with an embodiment of the invention;

FIG. 4 shows a graph of a user's movement precision under increased cognitive load compared to a normal trajectory, in accordance with an embodiment of the invention;

FIG. 5 shows a graph measuring movement deviation (calculated from the x-, y-positions) using data including the area under the curve (AUC), additional distance (AD), and maximum deviation (MD), in accordance with an embodiment of the invention; and

FIG. 6 shows a graph of mouse cursor speed (calculated from the x-, y-positions) under the influence of increased cognitive load and dissonance, in accordance with an embodiment of the invention.

### DETAILED DESCRIPTION OF THE PREFERRED EMBODIMENTS

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents that operate in a similar manner to accomplish a similar purpose. Several preferred embodiments of the invention are described for illustrative purposes, it being understood that the invention may be embodied in other forms not specifically shown in the drawings.

Modern computing technologies like workstations and laptops are equipped with an array of sensors to provide an enhanced user experience and to provide remarkable capabilities with the click of a mouse or the touch of a finger. In addition to providing tremendous capabilities and services, many of these sensors can be used to measure the motor movements of users with very fine detail and precision. For example, computer mice, touch pads, touch screens, keyboards, accelerometers, and so on, provide an array of data that can be collected at millisecond intervals. Our research team has shown that this data can be collected, analyzed and interpreted in near real-time, providing insights for a broad range of applications.

We believe that in order to secure data on the ever-eroding network boundary (e.g., EFSS and other FMS solutions), you need to capture end point data (e.g., behavioral events and movements) to identify anomalous events in near real-time. The cost for gaining this insight is low compared to the benefit that organizations may have gaining this new and novel signal. This process of detecting UDA via behavioral and movement anomalies can fundamentally change how organizations monitor, discover and mitigate UDA.

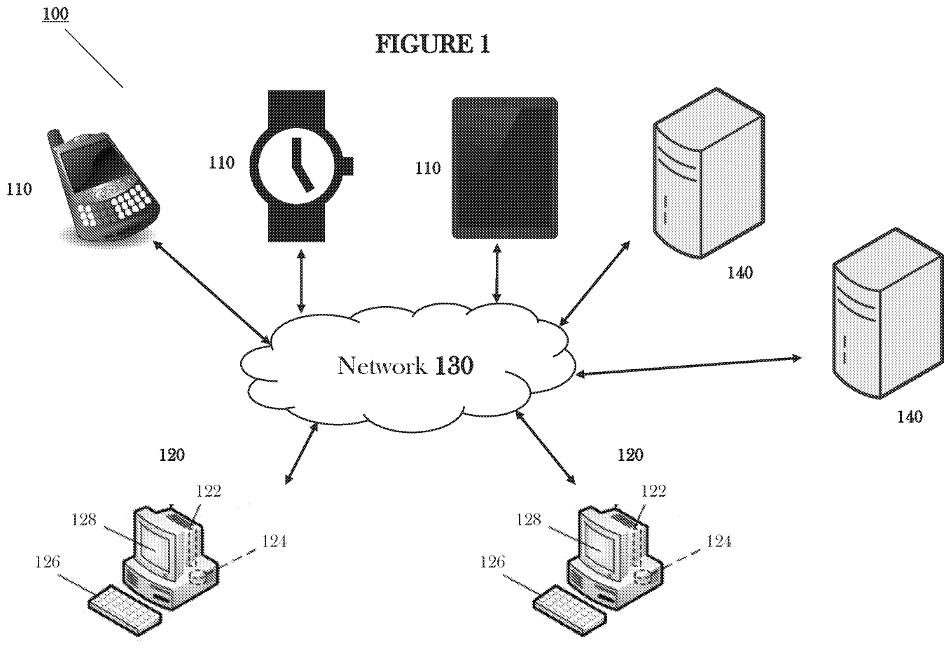

FIG. 1 is an exemplary embodiment of the hardware of the system. In the exemplary system 100, one or more peripheral devices 110 are connected to one or more computers 120 through a network 130. Examples of peripheral devices 110 include clocks, smartphones, tablets, wearable devices such as smartwatches, and any other networked devices that are known in the art. The network 130 may be a wide-area network, like the Internet, or a local area network, like an intranet. Because of the network 130, the physical location of the peripheral devices 110 and the computers 120 has no effect on the functionality of the hardware and software of the invention. Unless otherwise specified, it is contemplated that the peripheral devices 110 and the computers 120 may be in the same or in different physical locations. Communication between the hardware of the system may be accomplished in numerous known ways, for example using network connectivity components such as a modem or Ethernet adapter. The peripheral devices 110 and the computers 120 will both include or be attached to communication equipment. Communications are contemplated as occurring through industry-standard protocols such as HTTP.

Each computer 120 is comprised of a central processing unit 122, a storage medium 124, a user-input device 126, and a display 128. Examples of computers that may be used are: commercially available personal computers, open source computing devices (e.g. Raspberry Pi), commercially available servers, and commercially available portable devices (e.g. smartphones, smartwatches, tablets). In one embodiment, each of the peripheral devices 110 and each of the computers 120 of the system may have the software related to the system installed on it. In such an embodiment, data may be stored locally on the networked computers 120 or alternately, on one or more remote servers 140 that are accessible to any of the networked computers 120 through a network 130. The remote servers 140 may store databases comprising the file management systems that may be used by the disclosed invention. In alternate embodiments, the software may run as an application on the peripheral devices 110.

Most public and private organizations utilize a variety of Enterprise File Management Systems (FMS) to enable trusted employees and contractors access to vast troves of data stored in multiple locations. The analytic approach contemplates the ability to unobtrusively capture and analyze Human-Computer interaction (HCI) dynamics—finely grained streams of data reflecting how an individual interacts with a computer system using a variety of input devices—that are indicative of Unauthorized Data Access (UDA) events. While many of the examples will refer to mouse movements, the approach will also work using a broad range of HCI devices, including but not limited to: touch pads, touch screen, track balls, keyboards, gyrometer and accelerometer orientation and movement data from tablets and smartphones.

Modern computing devices are equipped with an array of sensors and HCI devices that can be used to capture and measure the motor movements of users with very fine detail and precision. For example, a computer mouse streams finely grained data at millisecond precision that can be translated into a large number of statistical data features reflecting changes in speed, targeting accuracy, and so on. We have developed deep expertise for automatically collecting and analyzing users' typing and mobile device interactions by embedding a small JavaScript library into a variety of online systems that acts as a “listener” to capture all movements and events. Once embedded, the script sends raw HCI device data—both movements and events—to a secure web service that can be stored and analyzed, potentially in near real-time.

Recent neuroscience research has unequivocally demonstrated that strong linkages exist between changes in cognitive processing (e.g., cognitive conflict, emotion, arousal, etc.) and changes in hand movements. When a person knowingly engages in a UDA event, they are more likely to experience cognitive or moral conflict. Likewise, malicious people are more likely to experience hesitations as they reconsider their planned and current actions. Such competing cognitions can influence one's fine motor control. For example, when moving the mouse to a specific file in order to engage in a UDA event, this person is much more likely to have cognitive or emotional changes due to thoughts related to the act itself as well as any related to aborting the illicit activity. Such thoughts will more likely result in less movement or typing precision, as compared to when the individual is acting purely in a non-fraudulent manner.

In addition to an increase in predictable movement anomalies, malicious individuals will also increase the likelihood of engaging in various behavioral events that are indicative of illicit acts. For example, a person engaging in UDA may have a vastly different pattern of behaviors than a person carefully performing their work-related duties. The malicious user, for example, may quickly open and close a sequence of files as they quickly search for desired information. On the other hand, a non-malicious person, will more carefully and deliberately navigate and select files to interact with.

The significance of this capability—the ability to infer behavioral intent by monitoring HCI dynamics—is profound. Recent neuroscience research has unequivocally demonstrated that strong linkages exist between cognitive processing (e.g., cognitive conflict, emotion, arousal, etc.) and hand movements. Motor movements were once thought to be the end-result of cognitive processing. However, a broad range of work has demonstrated that cognitive processing influences motor movements on an ongoing and continuous basis, even as mental processes are still unfolding. The “movement of the hand . . . offer continuous streams of output that can reveal ongoing dynamics of processing, potentially capturing the mind in motion with fine-grained temporal sensitivity . . . [revealing] hidden cognitive states that are otherwise not availed by traditional measures”. J. B. Freeman, R. Dale, and T. A. Farmer, “Hand in Motion Reveals Mind in Motion,” Frontiers in Psychology, vol. 2, no. 59, pp. 1-6, 2011. Mouse cursor tracking as a scientific methodology was originally explored as a cost-effective alternative to eye tracking to denote where people devote their attention in a human-computer interaction context. Dozens of studies have chosen mouse tracking for studying various cognitive and emotional processes. For example, viewing negative emotional images, increasing a person's stress level, viewing atypical information, and so on has been found to increase motor evoked potentials, hand and arm force production, and mouse movements.

The inventors have used mouse cursor tracking (and other human computer interaction [HCI] methods) to detect user states and characteristics, such as whether users are experiencing emotional arousal or valence, the level of ease-of-use a user experiences while interacting with a system, and the level of negative emotion individuals experience. Importantly, the inventors have also demonstrated that deceptive acts online can cause uncontrollable, yet measurable and predictable changes in people's mousing dynamics (how one moves their pointing device). As such, this approach may provide important and significant improvements when trying to detect unauthorized access to files within an EFSS or other FMS solutions.

It has previously been shown that mouse movements (as well as with other HCI devices) can accurately infer when a person is engaging in fraudulent activities, but the methods have been improved herein. Specifically, the research has shown that mouse movements can be used to detect states of arousal that are manifested by individuals who are off task or acting out of character. The inventors have also demonstrated that various types of stimuli can cause uncontrollable, yet measurable and predictable changes in people's HCI dynamics. Detecting these changes in HCI dynamics a well as changes in states of arousal and negative emotion will be the basis for the factors that will be used to detect purposeful unauthorized access.

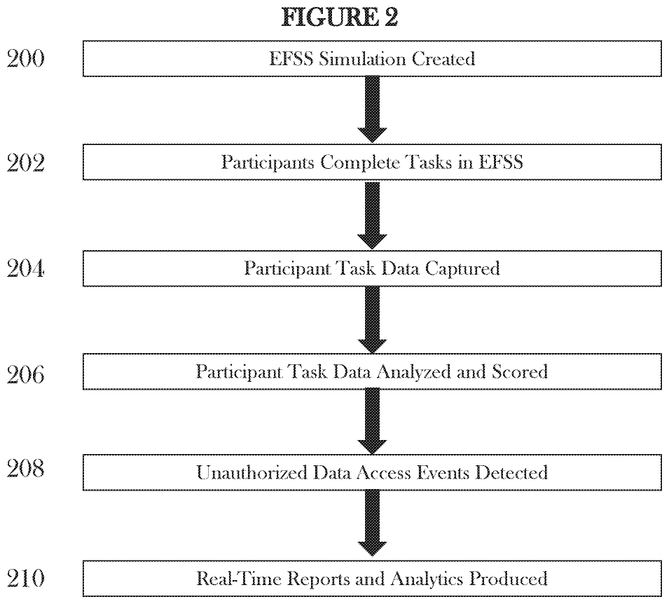

FIG. 2 discloses an exemplary software pathway in accordance an embodiment of the present invention. At step 200, a simulated EFSS that allows users to open, move, share, store files, create directories, and so on is created. The EFSS will also capture the timing and occurrence of events as well as measure mouse movements (as well as the data from other HCI devices depending on which are utilized by a particular human participant) of users while interacting with the system. Each participant is placed into a simulated organizational context (e.g., hospital, university, financial services, governmental agency, etc.) and be asked to preform common tasks a typical employee would be asked to complete (e.g., build directories, move and share files, open a particular file, gather some information from the file, record this information into a report, etc.). As the person performs the assigned task, their movements will be monitored and captured for later analysis at step 202. No proprietary or commercial software will be used in this system. Because the specific events and the movement data can be covertly collected using embedded JavaScript listener within the EFSS environment and the raw data is collected from common human-computer interaction (HCI) devices, this approach will provide a new signal for identifying possible UDA events.

After study participants complete a training session that will provide instructions on various aspects of operating the EFSS (e.g., making directories, moving and sharing flies, opening files to collect information, recording results, etc.), participants will be asked to complete a large number of fairly mundane tasks at step 202. When a person begins interacting with the EFSS, they will encounter a large number of files (e.g., 1000) in the root directory. They will then be asked to organize the files in a particular manner. Experimental tasks will include, but are not limited to the following: creating directories, creating subdirectories, moving files into directories (copying/cutting and pasting), sharing files with peers, opening files for viewing, searching on file types, and answering various questions about file content as well as various meta file characteristics.

At step 204, the FMS Sim will also capture the timing and occurrence of events as well as user HCI dynamics. Participants will operate in a simulated organizational context governmental agency) and be asked to perform various tasks typical of such employees (e.g., move and share files, gather information, update a report, etc.). Participants will also be required to agree to a Work Policy Agreement (WPA) to establish and explain expectations, guidelines, and rules when completing their assigned tasks (i.e., not to engage in any UDA events). As the person performs the assigned tasks, their HCI dynamics and behavioral patterns will be captured for later analysis.

While many of the tasks for participants to perform will be routine, some will be deliberately provocative. For example, participants will be asked to open files with “interesting” content (e.g., top secret information, salaries, employees on probation, provocative pictures, etc.) and confirm that the content inside the file indeed reflects the file name. Thus, if 2017-annualsalary.xls is provocative to a participant, it is much more likely that 2018annuasalary.xls, might be just as interesting (and more likely be opened—violating the WPA). In our prior work, we have designed a wide range of protocols that have been IRB approved for generating repeatable malicious behavior, with ground truth, in sub-populations of participants in a variety of context (e.g., theft, cheating, or unauthorized access), but this has not been in applied to an algorithm that will identify unauthorized access as it happens. When the user interacts with the FMS, all behavioral events and HCI dynamics will be logged for later analysis. Thus, we will capture finely grained data when a person completes an assigned task as well as when (or if) a person deviates from the assigned task (i.e., when engaging in a UDA event). This protocol will provide ground truth for both authorized and unauthorized events as well as related HCI dynamics and events (e.g., directory changes, tile opening, etc.). This data will be merged and organized around legitimate and illegitimate activities outcomes), so that machine learning can be used to identify meaningful movements and behavioral anomalies that infer malicious events.

It is envisioned that study participants will be asked to complete a series of mundane tasks that are typical of a modern work environment. Prior to engaging in the task, users will be required to agree to a WPA to establish and explain expectations, guidelines, and rules with regards to their interaction with the EFSS (i.e., not to engage in any UDA events). Users will then complete various data access, retrieval, and reporting tasks to simulate working within a large organization.

While people are completing tasks, we will have built in logic to monitor what people are doing and a database of scores, times, and so on, captured so that scoring the quality of the work can be quickly determined, as shown at step 206. Also, this database will capture WPA violations (e.g., opening unauthorized files). This will provide ground truth for both authorized and unauthorized events. Also, a person's interactions will be recorded so we can replay the interactions to create a library of typical behavioral and movement characteristics of authorized and unauthorized access. At the conclusion of the task, the participant will be told how they performed, be debriefed, and given participant compensation. In this manner, the EFFS simulator trained to be able to capture authorized and unauthorized events, both behaviors and movements, so that this raw data of both types of events can be captured with a diverse set of participants in various contexts. The trained algorithm can then be applied to a real-time operational environment. The ultimate goal of the EFSS environment is to allow real users the ability to choose (or not) to participant in UDA. By creating the simulated EFSS and having participants perform tasks where we can track actual behavior and movements, we believe we will be able to train an algorithm to detect authorized and unauthorized access, as shown in step 208.

There are various ways that UDA is dealt with today. First, most UDA technologies focus on mitigating data loss which include data encryption, remote backups, and media tagging so an insider cannot change or delete sensitive information. Many existing approaches do not detect UDA, but simply mitigate the downside risk of data loss. Recently, various artificial intelligence-based approaches for detecting insider threat UDA have become available. These approaches are primarily employing unsupervised machine learning algorithms on access logs. For example, such analyses include the examination of unusual geolocation, excessive data transmission, usual device or application access. Although promising, this technology relies on a post hoc analysis of the past. None of these approaches can provide a likelihood estimate of an individual's intention in near real-time while interacting with the EFSS or with other FMS solutions.

There are multiple use cases that are highly relevant to real world concerns for UDA. For example, one task context will relate to a military operating context; a second will focus on a contractor within a governmental security agency. Design studies in additional contexts including medical records, university records, and financial services may be conducted in order to identify any context relevant factors and to demonstrate the robustness of the approach. For each of these studies, a diverse population of young adults may be recruited to participate, around 500 per study.

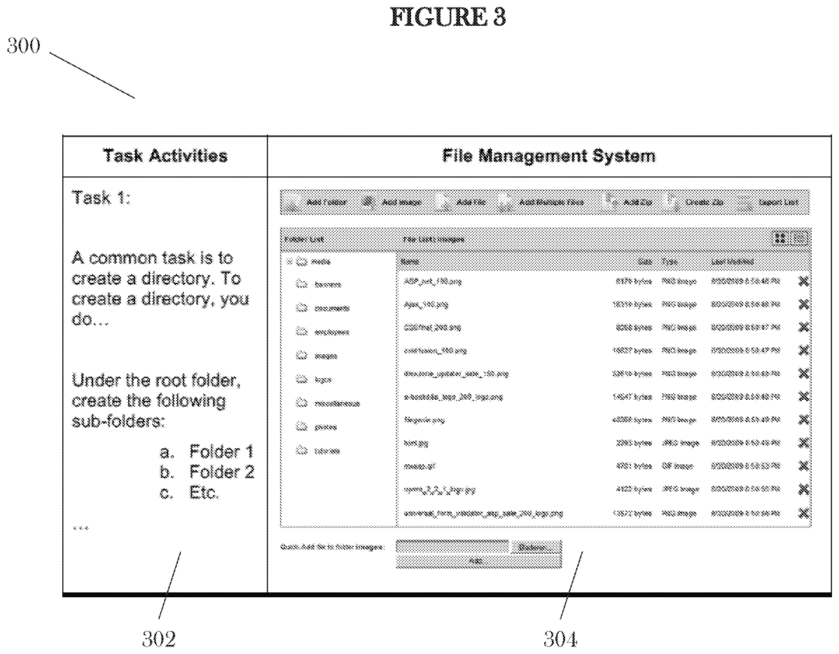

The simulated EFSS will have two panes on the computer display 300 as shown in FIG. 3. On the left, the task window 302, which will display a sequence of tasks for the user to perform. After the user completes a task, a new task will be loaded onto the task window. This will repeat until the simulated session is completed. On the right, the EFSS window, the simulated file management system 304 will be displayed and be used by participants to perform the assigned tasks. The EFSS simulator will operate manner and with a feature set similar to popular commercial systems.

Along with capturing behavioral events and tracking cursor movements, we will also collect perceptual measurements to aid in developing which personality or attitude factors best predict UDA. After honing in on the best set of factors for detecting UDA, we will train and test the accuracy of the predictive model for classifying survey attitudes and issues while also determining which real-time events and HCI dynamics data features best detect UDA. As shown at step 210, the predictive algorithm and training results in the following components:

FMS Simulator. This web-based solution will allow for viewing and management of files similar to other common EFSS (e.g., Dropbox, Box, etc.).

Tracking agent: This robust and lightweight JavaScript listening agent will track keyboard and navigation (e.g., mouse cursor) events that will be used for analysis.

Raw Data: Raw behavioral event and interaction data (HCI Dynamics) from human participants from all executed studies.

Replay Dashboard: a web-based dashboard where interactions between the FMS and the participant can be replayed for analysis.

Description of behavioral and interaction statistical features used in analysis.

Data analysis results and summary report.

### Predicting UDA Events

When a person knowingly engages in a UDA event, they are knowingly violating a Work Policy Agreement (WPA) (i.e., an agreement to only access the files and data needed to complete their assigned job-related duties). To explain how such malicious behavior correlates with predicable changes in HCI dynamics, we build on two axioms of maleficence and deception—cognitive dissonance (conflict) and cognitive load. First, when engaging in malicious activities, people experience cognitive or moral conflict. For example, due to guilt or the fear of being caught, deceptive people are more likely to experience hesitations as they reconsider their planned and current actions. Likewise, such individuals are much more likely to experience increased cognitive dissonance and conflict by questioning and reconsidering their planned fraudulent actions.

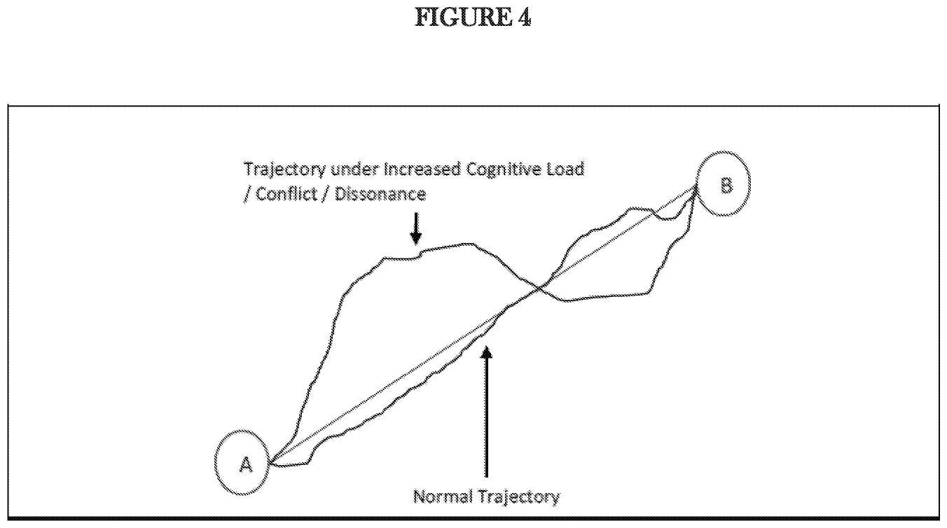

Such competing cognitions can influence one's fine motor control, as explained by the response activation model (RAM). Namely, the RAM posits that one's hand movements respond to all cognitions (i.e., thoughts) that have even a small potential to result in actual movement, so-called actionable potential. As such, when people knowingly engage in malicious activity, they are also more likely to deal with competing cognitions like double checking, reconsidering, hesitating, or questioning their actions. For example, when moving the mouse to a file in order to engage in a UDA event, whether motivated out of curiosity or malicious intent, this person is much more likely to have cognitive or emotional changes due to thoughts related to the act itself as well as any related to aborting the illicit activity. Such thoughts have actionable potential (e.g., to continue or stop)—even if the actions are not executed—resulting in less movement precision (i.e., increased variance in various interaction statistics), as compared when the individual is acting purely in a non-fraudulent manner, as shown in FIG. 4.

The RAM explains the relationship between cognitions and HCI dynamics: When a thought with actionable potential enters the mind (i.e., is in working memory), the mind automatically and subconsciously programs a movement response to fulfil that cognition's intention. This includes transmitting nerve pulses to the muscles to move hand and realize the intention (i.e., stop or move). These nerve impulses, in turn, ultimately result in hand movements toward the stimulus. If a person had accordant cognitions, their mouse trajectory would roughly follow a straight line to the movement's target (e.g., to the intended file to open in a FMS). Deviations from that straight line can result from competing cognitions due to being malicious—i.e., the mind programs movement responses toward other stimuli with actionable potential. Those deviations can also be captured by characteristics of the HCI dynamics data (e.g., mouse movements).

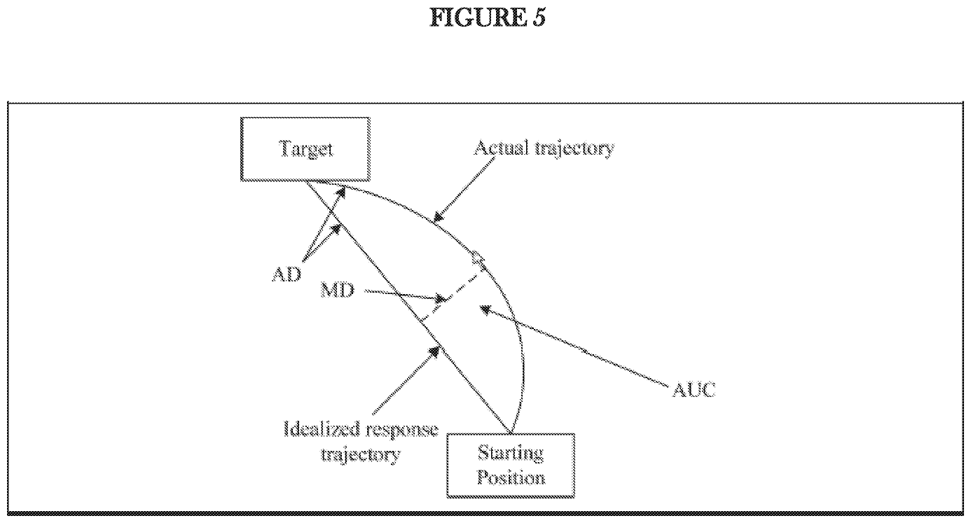

Second, deception is a complex cognitive process that increases cognitive load, another axiom of deception. When people are malicious they typically attempt or consider ways to minimize any evidence of their act. Such strategic behavior—i.e., manage information to appear truthful—increases cognitive load, thereby decreasing available working memory. When working memory is decreased, people's reaction times also become slower, and so do hand movements. Namely, when visually guiding the hand to a target, the brain has less time to program corrections to one's movement trajectory. Those corrections result in greater deviations from one's intended trajectory. In other words, movement precision decreases and such changes can be captured using a variety of data features from the raw data stream. This is shown in FIG. 5, which measures movement deviation using data including the area under the curve (AUC), additional distance (AD), and maximum deviation (MD).

One way the brain automatically compensates for decreased precision is to reduce the speed of movements. Hand (e.g., mouse) movement speed and precision are inversely related, so movement precision can only increase if the brain reduces movement speed. In other words, as the body has more time to perceive and program needed corrections, it allows the hand to operate more optimally within the restriction of slower reaction times. Thus, individuals engaging in malicious activities will have HCI dynamics that increase in movement deviations (i.e., X- and Y-axis flips, less efficient movements, changes in acceleration, and so on) as well as reductions in overall speed. In other words, engaging in UDA events will increase the likelihood that various movement anomalies will occur as compared to the movements associated with legitimate activities.

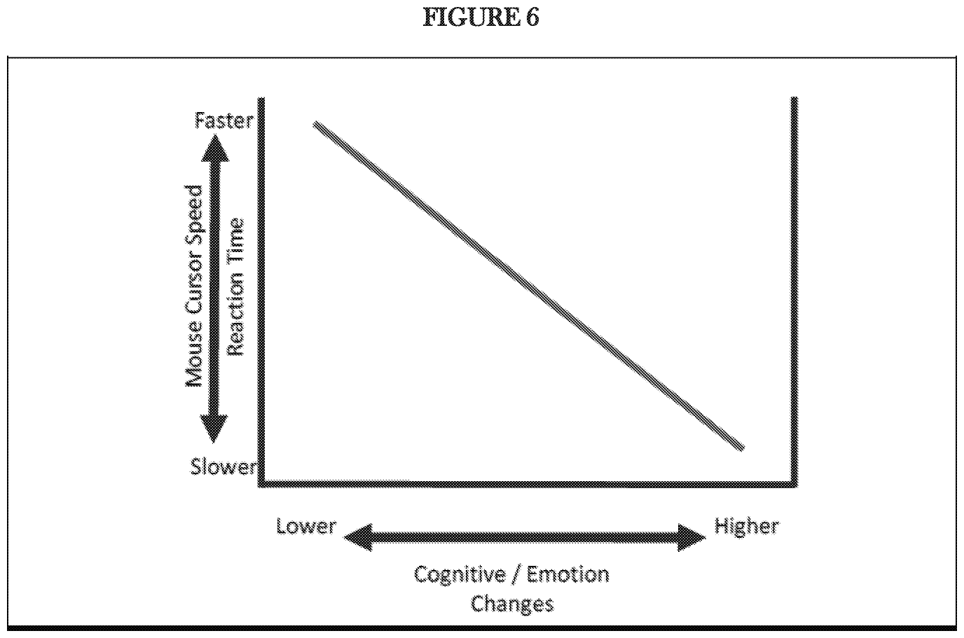

In addition to an increase in predictable movement anomalies, malicious individuals will also increase the likelihood of engaging in various behavioral anomalies that are indicative of illicit acts. A meaningful behavioral anomaly refers to an action that is more likely to be associated with a malicious event (i.e., suspicious behavior). For example, a person engaging in UDA may have a vastly different pattern of behaviors than a person carefully performing their work-related duties. The malicious user, for example, may quickly open and close a sequence of files as they quickly search for desired information. On the other hand, a non-malicious person, will more carefully navigate and select files to interact with. As part of our proposed research, we will identify and catalog such behavioral indicators associated with legitimate and illegitimate file access. Thus, individuals engaging in malicious activities are more likely to have behavioral anomalies indicative of NDA events. FIG. 6, reflects this, showing an exemplary graph of mouse cursor speed under the influence of increased cognitive load and dissonance.

The foregoing description and drawings should be considered as illustrative only of the principles of the invention. The invention is not intended to be limited by the preferred embodiment and may be implemented in a variety of ways that will be clear to one of ordinary skill in the art (i.e., using a broader range of HCI devices beyond a computer mouse). Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention.

1. A computer-implemented method for detecting unauthorized data access comprising the steps of:
compiling a database of user behaviors associated with unauthorized data access;
detecting a user's behavior on a networked computing device;
scoring the user's behavior based on cognitive dissonance and cognitive load against the database of user behaviors associated with unauthorized data access; and
transmitting a notification based on the scoring that the user's behaviors are indicative of unauthorized data access.

2. The computer-implemented method of claim 1, wherein an EFSS simulation is created to compile the database of user behaviors.

3. The computer-implemented method of claim 1, further comprising the step of outputting one or more real-time reports or analytics on user behavior on the networked computing device.

4. computer-implemented method of claim 1, wherein the user behavior detected is mouse cursor trajectory.

5. The computer-implemented method of claim 1, wherein the user behavior detected is mouse cursor speed associated with the cognitive load score.

6. The computer-implemented method of claim 1, wherein the user's behavior is detected through a human-computer interaction (HCI) device.

7. The computer-implemented method of claim 6, wherein the user's behavior is quantified as actionable potential from the HCI device.

8. The computer-implemented method of claim 1, wherein the scoring is indicative of behavioral anomalies.

9. The computer-implemented method of claim 8, wherein the behavioral anomalies are calculated as a deviation from a straight line to a target on the networked computer device's screen.

10. computer-implemented method of claim 1, wherein the scoring applies the response activation model (RAM).

11. A system for detecting unauthorized data access comprised of one or more peripheral devices, a network, one or more networked computers, and one or more remote servers, wherein the one or more remote servers, peripheral devices, and/or the one or more networked computers are configured to:
compile a database of user behaviors associated with unauthorized data access;
detect a user's behavior on the one or more networked computers;
score the user's behavior based on cognitive dissonance and cognitive load against the database of user behaviors associated with unauthorized data access; and
transmit a notification based on the scoring that the user's behaviors are indicative of unauthorized data access.

12. The system of claim 11, wherein an EFSS simulation is created to compile the database of user behaviors.

13. The system of claim 11, wherein the one or more remote servers output one or more real-time reports or analytics on user behavior on the networked computing device.

14. The system of claim 11, wherein the user behavior detected is mouse cursor trajectory.

15. The system of claim 11, wherein the user behavior detected is mouse cursor speed associated with a cognitive load score.

16. The system of claim 11, wherein the user's behavior is detected through a human-computer interaction (HCI) device.

17. The system of claim 16, wherein the user's behavior is quantified as actionable potential from the HCI device.

18. system of claim 11, wherein the scoring is indicative of behavioral anomalies.

19. The system of claim 18, wherein the behavioral anomalies are calculated as a deviation from a straight line to a target on the networked computer device's screen.

20. The system of claim 11, wherein the scoring applies the response activation model (RAM)
